(19) United States
(12) Patent Application Publication
(10) Pub. No.: US 2026/0345393 A1
(43) Pub. Date: Nov. 5, 2026

(54) EAR CLEANER

(71) Applicant: OSCIMED SA, La Chaux-de-Fonds (CH)

(72) Inventors: Jacques MAGNIN, La Chaux-de-Fonds (CH); Pascal CHOLLET

(21) Appl. No.: 19/125,412

(22) PCT Filed: Oct. 18, 2023
(86) PCT No.: PCT/EP2023/078923

§ 371 Date: Apr. 29, 2025

(30) Foreign Application Priority Data
Nov. 7, 2022 (CH) CH001324/2022

(51) Int. Cl. A61F 11/00

(52) U.S. Cl. CPC A61F 11/006

(57) ABSTRACT

An ear cleaner includes a handle having a generally rotationally symmetrical shape about an axis of revolution defining the axis of rotation during cleaning of the ear and formed by the intersection of a frontal plane and a median plane, and including, at at least one end of the handle, a cleaning tip coaxial with the handle. The tip includes a cylindrical body coaxial with the handle and having, on a distal portion, a head including fins arranged radially on the outer periphery of the distal portion of the body, each fin extending along the distal portion along the axis of revolution and having a cross-section that gradually increases from the distal end of the head. The tip includes a scoop provided at the distal end of the head, in the continuity of the head, and having at least one functional surface substantially parallel to the frontal plane.

### TECHNICAL FIELD

The present invention relates to an ear cleaner comprising a handle having a generally rotationally symmetrical shape about an axis of revolution defining the axis of rotation during the cleaning of the ear and comprising, at at least one end of the handle, a cleaning tip coaxial with the handle.

### PRIOR ART

The usual process for cleaning ears is to utilise cotton buds, short sticks covered at each end with a small ball of cotton. However, the disadvantage of cotton buds is that they push the earwax further into the ear canal. This can eventually create plugs of earwax.

Furthermore, disposable cotton buds must be replaced by more environmentally friendly products, while remaining effective for cleaning ears.

To this end, cotton buds made of washable plastic have been developed. However, they still have the same disadvantage of pushing earwax into the ear canal.

Different variants of ear cleaners are also offered. The ear cleaners are less invasive than a cotton bud and can generally be cleaned in order to be reused. For example, there exist ear cleaners with a tip having spiral grooves, or a cross-shaped tip that forms a spiral in the ear canal during rotation. These tips are made of soft and flexible silicone for comfort in the ear. However, the disadvantage of these is that they do not have a sufficiently effective mechanical action, as the tip is too soft.

Metal ear cleaners having heads of different shapes are also offered: metal wire wound in a spiral, spoon, rings, etc. However, users may be a little apprehensive about introducing a metal rod into their ear canals.

The cotton buds or ear cleaners available on the market are therefore not satisfactory.

It is therefore necessary to be able to offer a more efficient ear cleaner, making it possible to extract earwax effectively, without causing any damage to the ear canal.

### DISCLOSURE OF THE INVENTION

For that purpose, the invention relates to an ear cleaner comprising a handle having a generally rotationally symmetrical shape about an axis of revolution defining the axis of rotation during the cleaning of the ear and formed by the intersection of a frontal plane and a median plane, and comprising, at at least one end of the handle, a cleaning tip coaxial with the handle.

According to the invention, said tip comprises a cylindrical body coaxial with the handle and having, on a distal portion, a head comprising a series of fins arranged radially on the outer periphery of the distal portion of the body, each fin extending along said distal portion along the axis of revolution and having a cross-section that gradually increases from the distal end of the head, giving said head an arcuate shape, said tip also comprising a scoop provided at the distal end of the head, in the continuity of the head, said scoop having at least one functional surface which is substantially parallel to the frontal plane.

The scoop advantageously has a cross-section that gradually increases from its distal end up to the distal end of the head.

Each fin preferably has a cross-section gradually increasing from the distal end of the head then gradually reducing up to the proximal end of the head.

The body of the tip preferably has an outer diameter that gradually increases from the distal end of the distal portion of the body, giving a conical shape.

Thus, the ear cleaner according to the invention is very effective, making it possible to capture the earwax without pushing it into the ear canal thanks in particular to the gradual conical shape of the fins, giving the head an arcuate shape, to the gradual conical shape at the end of the body of the tip, and to the reduced section of the scoop integrated at the end of the head.

### BRIEF DESCRIPTION OF THE DRAWINGS

Other characteristics and advantages of the present invention will appear on reading the following detailed description of an embodiment of the invention, which is provided by way of a non-limiting example, and with reference to the appended drawings, wherein:

FIG. 1 shows a top view of an ear cleaner according to the invention;

FIG. 2 shows an isometric side view of the ear cleaner of FIG. 1, the different tips being separated from the handle;

FIG. 3 shows an isometric top view of the ear cleaner of FIG. 1, the different tips being separated from the handle;

FIG. 4 shows a sectional view along the median plane of the ear cleaner of FIG. 1;

FIG. 5 shows an isometric view of a tip used in the ear cleaner of the invention;

FIG. 6 shows a sectional view along the median plane of the tip of FIG. 5;

FIG. 7 shows a cross-sectional view of the tip of FIG. 5; and

FIG. 8 shows a front view of the tip.

### EMBODIMENTS OF THE INVENTION

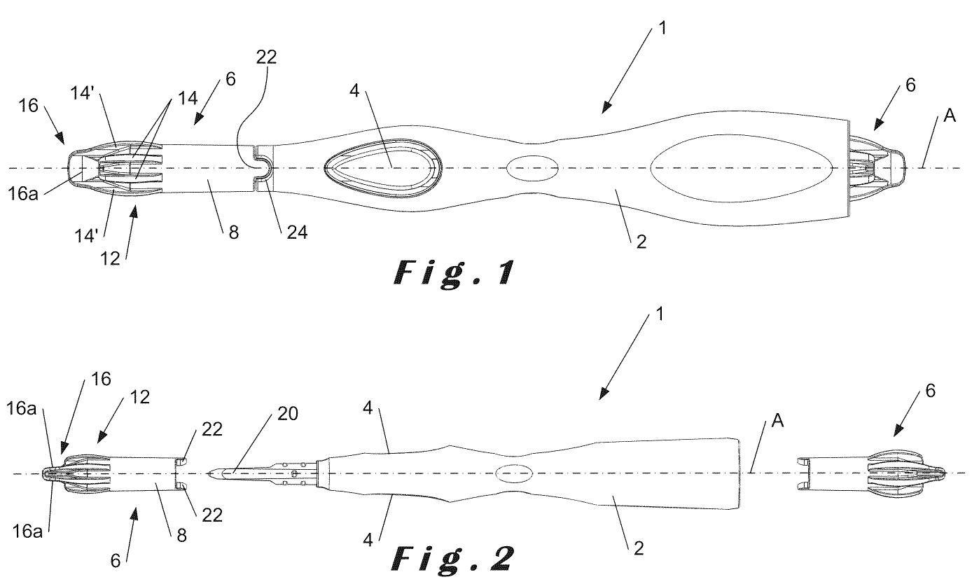

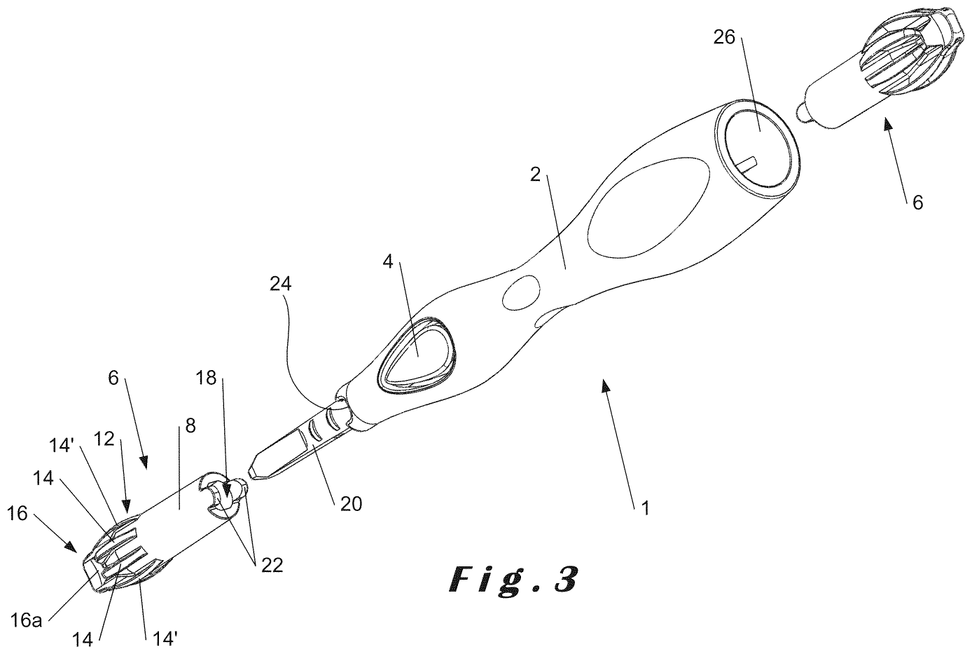

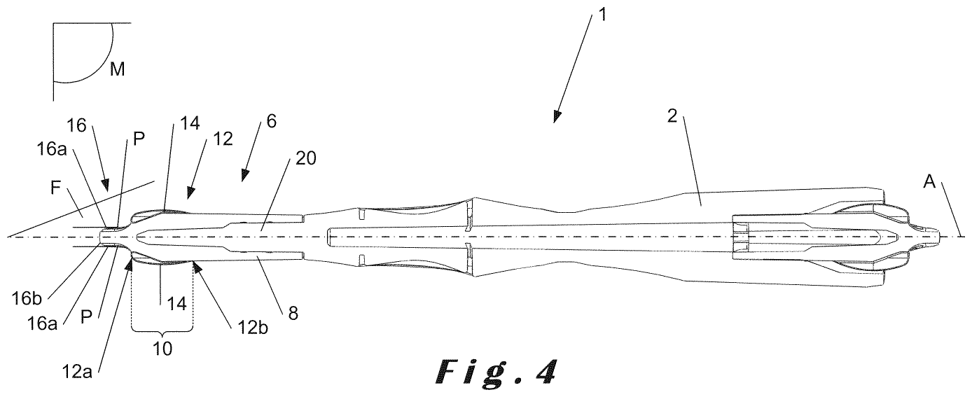

With reference to FIGS. 1 to 4, the present invention relates to an ear cleaner 1 comprising a handle 2 having a generally rotationally symmetrical shape about a longitudinal axis of revolution A defining the axis of rotation during the cleaning of the ear. The axis A is formed by the intersection of a frontal plane F and a median plane M, shown in FIG. 4.

The diameter of the handle 2 can vary in order to give it an ergonomic shape. Furthermore, two flats 4 parallel to the frontal plane F are provided, the role of which will be described in more detail below.

The ear cleaner 1 also comprises, at at least one end of the handle 2, a cleaning tip 6 coaxial with the handle 2, arranged to be able to be assembled integrally with the handle 2.

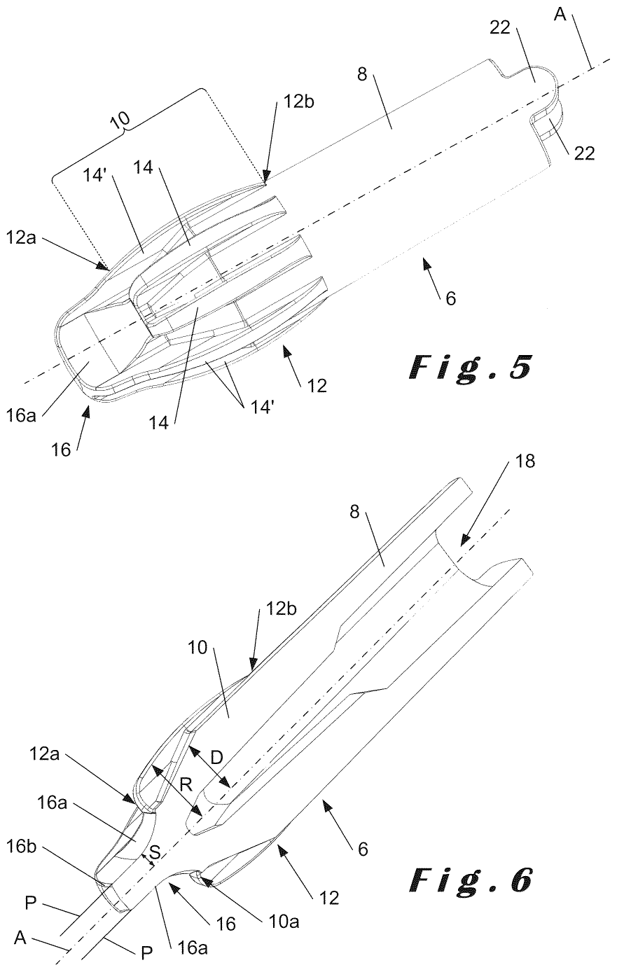

According to the invention, and with reference more particularly to FIGS. 5 to 8, said tip 6 comprises a cylindrical body 8 coaxial with the handle 2, arranged to be able to be integrally assembled with the handle 2, and having, on a distal portion 10 (i.e. at the end of the body 8 opposite the handle 2), a head 12 comprising a series of solid fins 14, arranged radially and distributed regularly on the outer periphery of the distal portion 10 of the body 8, each fin 14 extending along said distal portion 10 along the axis of revolution A. The head 12 thus has a rotating function, each fin 14 making it possible to extract the earwax by turning on the walls of the ear canal.

In the example shown, the tip comprises eight fins 14, but it is obvious that the number of fins can vary.

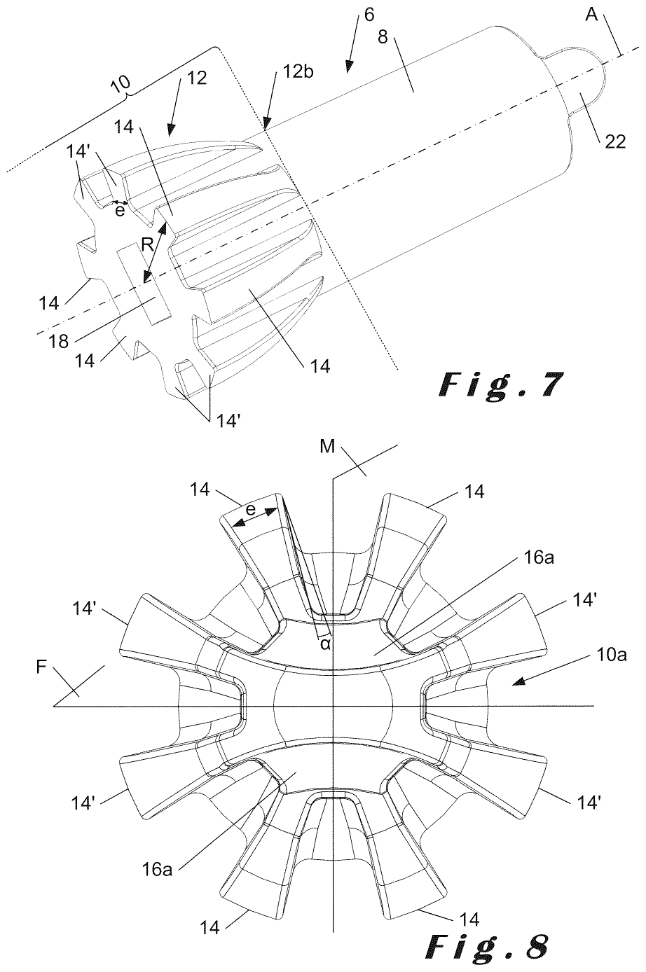

Each fin 14 advantageously has a cross-section (i.e. in a plane perpendicular to the axis A) that gradually increases from the distal end 12a of the head 12, giving said head 12 an arcuate shape. Thus, the outer radius R (cf. FIGS. 6 and 7) of each fin 14, defining its height seen in cross-section in a radial plane, gradually increases from the distal end 12a of the head 12. This makes it possible to not push earwax into the ear canal, and to facilitate the insertion of the tip into the ear canal.

In addition, said tip 6 comprises a scoop 16 provided at the distal end 12a of the head 12, in the continuity of said head 12, said scoop 16 having at least one functional surface 16a extending in a plane P which is substantially parallel to the frontal plane F (cf. FIGS. 4 and 6). This means that the functional surface 16a can be flat, parallel to the frontal plane F, or that it can be slightly curved in order to form, in front view, a concave-shaped cross-section. The angle of curvature of the functional surface 16a with respect to the frontal plane F can be, for example, between 0° (flat functional surface 16a parallel to the frontal plane F) and 30° (curved functional surface 16a), preferably between 10° and 25°.

Due to the symmetry of the tip 6, the scoop 16 preferably comprises two functional surfaces 16a extending on either side of the frontal plane F.

The scoop 16 advantageously has, on either side of the frontal plane F, in front view, a concave-shaped cross-section, preferably symmetrical with respect to said frontal plane F, giving said scoop 16 an outer biconcave appearance as it appears in FIG. 8. This biconcave shape of the scoop 16, with an angle of curvature with respect to the frontal plane F of less than 30°, and preferably between 10° and 25°, very advantageously makes it possible to reduce the impact in the earwax and to have a better scraping effect. It is obvious that, in another variant which is not shown, the scoop could have a curved functional surface and a flat functional surface, or a functional surface of different curvature.

The scoop 16 advantageously has a cross-section S (cf. FIG. 6) that gradually increases from its distal end 16a up to the distal end 12a of the head 12. Thus, the distal end 16a of the scoop 16 has the thinnest possible section in order to join the end of the head 12, sloping gently. The small section of the scoop 16 makes it possible to extract the earwax by scraping inside the ear without pushing the earwax deep inside the ear canal.

Each fin 14 preferably has, along the axis A, a cross-section (i.e. defined by the outer radius R in cross-section in a radial plane) gradually increasing from the distal end 12a of the head 12 then gradually reducing up to the proximal end 12b of the head 12 (cf. FIGS. 6 and 7). This means that each fin 14 has a height, taken from the base of the fin 14 in a radial plane, that gradually increases from the distal end 12a of the head 12 that then gradually reduces up to the proximal end 12b of the head 12 along the axis A. This makes it possible to avoids sharp edges for user comfort.

Each fin 14 preferably has a positive rake angle a, for example, between 0.5° and 20°.

Furthermore, each fin 14 has, in a transversal plane, a shape widening in the direction of the exterior, each fin 14 having a thickness e that gradually increases from the base of the fin 14 (connecting the distal portion 10 of the body 8) in the direction of the exterior in a same transversal plane (cf. FIGS. 7 and 8). The fin 14, taken in a transversal plane, can widen at an angle of between 0.5° and 45° with respect to the axis of symmetry of the fin 14 in the transversal plane. This makes it possible to achieve a better scraping effect which does not push earwax into the ear canal.

The head 12 advantageously comprises, on a same side of the frontal plane F, and on each side of said frontal plane F in symmetry with respect to said frontal plane F, two lateral fins, referenced more specifically 14′ in the figures, that extend beyond the distal end 12a of the head 12 in order to join one another, giving the scoop 16 its outer biconcave appearance and forming, in the centre, one of the curved functional surfaces of the scoop 16. The other characteristics which are described above in a general manner for the fins 14 also apply to the fins referenced 14′.

The body 8 of the tip 6 preferably has an outer diameter D that gradually increases from the distal end 10a of the distal portion 10 of the body 8, giving the distal end of the body 8 that widens from the scoop 16 a conical shape. In other words, the outer diameter D of the end of the distal portion 10 on the side of the scoop 16 gradually reduces in the direction of said scoop 16. The conical shape of the distal end of the body 8 from the scoop 16 makes it possible to not push the earwax deep inside the ear canal.

The tip 6, that is to say the body 8, the fins 14 and the head 16, is preferably made of a single material, for example by moulding. Such a material can be, for example, a silicone, an EVA (ethylene vinyl acetate), a TPE (thermoplastic elastomer) or a PU (polyurethane), or any other suitable material.

The body 8 of the tip 6 advantageously comprises a hollow core 18 (cf. FIG. 6) arranged to receive a longitudinal framework 20 provided at one end of the handle 2 in order to mount the tip 6 on the handle 2 by inserting the framework 20 into the core 18. The shape of the framework 20 corresponds to the shape of the core 18 in order to ensure that the tip 6 is securely held on the handle 2.

The framework 20 preferably extends approximately up to the distal end 10a of the distal portion 10 of the body 8. The framework 20 is advantageously made of a more rigid material than the body 8 in order to constitute a rigid skeleton for the tip 6, making possible better insertion into the ear canal. The handle 2 and its framework 20 can be made, for example, of POM (polyoxymethylene), PET (polyethylene terephthalate), PP (polypropylene), PA (polyamide), or any other suitable material.

The tip 6 is advantageously arranged to be mounted on the handle 2 in a removable manner so that it can be replaced by a tip of a different size or by a new tip 6.

In order to ensure that the tip 6 is locked on the handle 2, locking means such as tabs 22, arranged to engage in corresponding notches 24 provided on the handle 2, can be provided, for example, at the proximal end of the body 8.

The tip 6 and the handle 2 advantageously comprise indexing means arranged to assemble the tip 6 and the handle 2 such that the main plane P of the functional surfaces 16a of the scoop 16 is parallel to the frontal plane F, once the tip 6 is mounted on the handle 2.

In a preferred embodiment, the locking means, such as the tabs 22, and the corresponding notches 24, are positioned respectively on the periphery of the tip 6 and the periphery of the handle 2 so as to also constitute the indexing means. Thus, the tip 6 is automatically positioned correctly with respect to the handle 2 during the assembly. For this reason, the flats 4 parallel to the frontal plane F are also parallel to the main plane P of the functional surfaces 16a of the scoop 16, once the tip 6 is mounted on the handle 2. Thus, the handle 2 comprises a cylindrical gripping zone, which is constituted by the cylindrical part of the handle 2, and a flat gripping zone which is parallel to the frontal plane F and to the main plane P of the functional surfaces of the scoop, said flat gripping zone being constituted by the flats 4.

The cylindrical gripping zone makes handling with the fingers possible in order to easily rotate the handle 2 during use of the fins 14 for rotary cleaning. The flat gripping zone makes handling with the fingers possible in order to guarantee that the functional surfaces of the scoop 16 are correctly oriented during the use thereof.

It is obvious that, in another embodiment, the tip 6 can be mounted in a non-removable manner on the handle 2.

The length of the body 8 of the tip 6 is chosen so as to correspond to a safety length to avoid losing the tip 6 in the ear in the event of the handle accidentally breaking or in the event of accidental unlocking.

The handle 2 advantageously comprises, at one end, a cleaning tip 6 and, at the other end, an inner housing 26 arranged to receive at least one other cleaning tip 6. The two cleaning tips 6 can be of the same size or a different size. In the example shown, the inner housing 26 is dimensioned to reveal in particular the scoop 16 and a portion of the head 12 of the tip 6. The base of said inner housing 26 has a plinth on which the body 8 of the tip 6 is clipped, ensuring that the tip 6 is held in the housing 26. In another variant which is not shown, the inner housing 26 can be dimensioned to be able to fully house a tip 6 therein, said housing 26 then being closed by a plug (not shown).

The ear cleaner 1 is preferably offered with two tips 6 of different size.

The ear cleaner 1 according to the invention has a dual function of extraction and scraping thanks to the fins 14 and the scoop 16. The rotating function of the fins 14 makes it possible to extract the earwax by turning, without spiral deformation, on the walls of the ear canal via the rotation of the cylindrical gripping zone of the handle 2. The scoop 16, which is correctly oriented thanks to the flat gripping zone, makes it possible to scrape inside the ear. The scoop 16 is self-guided thanks to the arcuate shape of the head 12 which makes it possible to create a ball joint and centring effect for said scoop 16.

The gradual conical shape of the fins 14, giving the head 12 an arcuate insertion shape, the gradual conical shape of the distal end of the body 8 of the tip 6, as well as the reduced section of the scoop 16 make it possible to capture the earwax and extract it without pushing it into the ear canal.

It is obvious that, according to another possible embodiment, the ear cleaner can comprise a handle 2 having a generally rotationally symmetrical shape about an axis of revolution A defining the axis of rotation during the cleaning of the ear and formed by the intersection of a frontal plane F and a median plane M, and comprising, at at least one end of the handle 2, a cleaning tip 6 coaxial with the handle 2, in which said tip 6 comprises a cylindrical body 8 coaxial with the handle 2 having, on a distal portion 10, a head 12 having a suitable configuration which is different from the fins described above, and in which said tip 6 comprises a scoop 16 provided at the distal end 12a of the head 12, in the continuity of said head 12, said scoop 16 having at least one functional surface 16a which is substantially parallel to the frontal plane F, that is to say flat or curved, as described above. The scoop 16 preferably has, on either side of the frontal plane F, in the front view, a concave-shaped cross-section which is preferably symmetrical with respect to said frontal plane F, giving said scoop 16 an outer biconcave appearance as it appears in FIG. 8. All the other characteristics described above can be combined with this embodiment.

The ear cleaner 1 according to the invention is reusable since the tips 6 can be easily removed for cleaning.

1. An ear cleaner comprising:
a handle having a generally rotationally symmetrical shape about an axis of revolution defining the axis of rotation during cleaning of an ear and formed by an intersection of a frontal plane and a median plane, the handle comprising, a cleaning tip coaxial with the handle at at least one end of the handle, said tip comprising
a cylindrical body coaxial with the handle and having, on a distal portion, a head comprising a series of fins disposed radially on an outer periphery of said distal portion of the cylindrical body, each of the fins extending along said distal portion along the axis of revolution and having a cross-section that gradually increases from a distal end of the head, giving the head an arcuate shape, and
a scoop provided at the distal end of the head, in a continuity of said head, said scoop having at least one functional surface which is substantially parallel to the frontal plane.

2. The ear cleaner according to claim 1, wherein the scoop has a cross-section that gradually increases from a distal end of the scoop up to the distal end of the head.

3. The ear cleaner according to claim 1, wherein each of the fins has a cross-section gradually increasing from the distal end of the head then gradually reducing up to a proximal end of the head.

4. The ear cleaner according to claim 1, wherein each of the fins has a positive rake angle.

5. The ear cleaner according to any claim 1, wherein each of the fins has, in a transversal plane, a shape widening in a direction of the exterior.

6. The ear cleaner according to claim 1, wherein the cylindrical body the tip has an outer diameter that gradually increases from the distal end of the distal portion of the cylindrical body, giving a conical shape.

7. The ear cleaner according to claim 1, wherein the cylindrical body of the tip comprises a hollow core configured to receive a framework provided at one end of the handle in order to mount the tip on the handle.

8. The ear cleaner according to claim 1, wherein the tip is configured to be mounted on the handle in a removable manner.

9. The ear cleaner according to any one of the preceding claims claim 1, wherein the tip and the handle comprise an index system configured to assemble the tip and the handle such that the at least one functional surface of the scoop is parallel to the frontal plane.

10. The ear cleaner according to claim 1, wherein a material of the body is made more rigid than a material of the fins and a material of the head.

11. The ear cleaner according to claim 1, wherein the handle comprises a cylindrical gripping zone and a flat gripping zone parallel to the frontal plane.

12. The ear cleaner according to claim 1, wherein the handle comprises, cleaning tip at one end thereof and an inner housing at the other end thereof, the inner housing being configured to receive another cleaning tip.

13. The ear cleaner according to 1, wherein the at least one functional surface of the scoop comprises two functional surfaces extending on either side of the frontal plane.

14. The ear cleaner according to claim 1, wherein the scoop has, on either side of the frontal plane, a concave-shaped cross-section, giving said scoop a biconcave appearance.

15. The ear cleaner according to claim 1, wherein the head comprises, on a same side of the frontal plane, two lateral fins that extend beyond the distal end of the head in order to join one another, forming the at least one functional surface of the scoop.
